(19) United States
(12) Patent Application Publication
(10) Pub. No.: US 2012/0219459 A1
(43) Pub. Date: Aug. 30, 2012

(54) PHOTOCATALYTIC STRUCTURAL MEMBER AND DEODORIZING DEVICE

(75) Inventor: Morio Nakatani, Ichinomiya-City (JP)

(73) Assignee: Sanyo Electric Co., Ltd., Moriguchi-shi (JP)

(21) Appl. No.: 13/505,256

(22) PCT Filed: Aug. 24, 2010
(86) PCT No.: PCT/JP2010/064223

§ 371 Date: Apr. 30, 2012

(30) Foreign Application Priority Data
Oct. 30, 2009 (JP) 2009-249907

(51) Int. Cl. A61L 9/00

(52) U.S. Cl. 422/107; 422/121; 422/120

(57) ABSTRACT

A photocatalytic structural member is provided with a substrate having a concave-convex structure formed on a surface thereof, and a photocatalyst film which is disposed on a side of the concave-convex structure of the substrate and reflects a shape of the concave-convex structure. The concave-convex structure is formed with a pitch smaller than a wavelength of light which causes a photocatalytic reaction on the photocatalyst film. A deodorizing device is provided with the photocatalytic structural member, a light source which emits the light from a side of the substrate to the photocatalytic structural member, and a fan which causes an air to flow to the photocatalytic structural member.

### TECHNICAL FIELD

The present invention relates to a photocatalytic structural member which causes a photocatalytic reaction by irradiation of light of a predetermined wavelength, and a deodorizing device incorporated with the photocatalytic structural member and for purifying a material to be purified that is contained in the air.

### BACKGROUND ART

In recent years, development has progressed on photocatalytic devices which perform air purification, deodorization, water purification, antibacterial treatment, soil release, water decomposition, with use of a photocatalytic structural member containing a photocatalytically active material. Generally, the photocatalytic structural member of this kind is formed by laminating a photocatalyst film composed of titanium oxide (TiO₂) or the like on a.

### SUMMARY OF INVENTION

### Technical Problem

In the photocatalytic structural member, light (e.g. ultraviolet light) of a predetermined wavelength is irradiated onto the photocatalyst film, whereby a material adhered to a surface of the photocatalyst film is purified by a photocatalytic action. In this case, it is desirable to efficiently utilize the light for irradiation in a photocatalytic reaction on the photocatalyst film.

However, if light for causing a photocatalytic reaction is irradiated from the photocatalyst film side, the light is blocked by the material to be purified that is adhered to the surface of the photocatalyst film. This may obstruct the light from impinging on the photocatalyst film. On the other hand, when light for causing a photocatalytic reaction is irradiated from the substrate side, the light is reflected on the interface between the substrate and the photocatalytic film. This may obstruct the light from impinging on the photocatalyst film.

In view of the above problems, an object of the invention is to provide a photocatalytic structural member that enables to efficiently cause a photocatalytic reaction.

Another object of the invention is to provide a novel deodorizing device incorporated with the photocatalytic structural member.

### Solution to Problem

A photocatalytic structural member according to a first aspect of the invention is provided with a substrate having a concave-convex structure formed on a surface thereof; and a photocatalyst film which is disposed on a side of the concave-convex structure of the substrate and reflects a shape of the concave-convex structure. In this arrangement, the concave-convex structure is formed with a pitch smaller than a wavelength of light which causes a photocatalytic reaction on the photocatalyst film.

A deodorizing device according to a second aspect of the invention is provided with the photocatalytic structural member according to the first aspect, a light source which emits the light from a side of the substrate to the photocatalytic structural member, and a fan which causes an air to flow to the photocatalytic structural member.

### BRIEF DESCRIPTION OF DRAWINGS

FIG. 1 is a diagram showing an arrangement of a purification plate in an embodiment;

FIG. 2 is a diagram for describing a sequence of forming a substrate in the embodiment;

FIG. 3 shows a measurement result about a reflectance lowering action of a concave-convex structure in the embodiment;

FIG. 4 is a diagram showing an arrangement of a deodorizing device in the embodiment;

FIG. 5 is a diagram for describing a control to be performed by a control device in the embodiment;

FIG. 6 is a diagram for describing a modification of the arrangement of the deodorizing device, and a modification of the control to be performed by the control device in the embodiment;

FIG. 7 is a diagram showing modifications on an arranged position of the purification plate in the embodiment; and

FIG. 8 is a diagram showing modifications of a pulse signal and an emission power of a semiconductor laser in the embodiment.

### DESCRIPTION OF EMBODIMENTS

In the following, an embodiment of the invention is described referring to the drawings.

<Purification Plate>

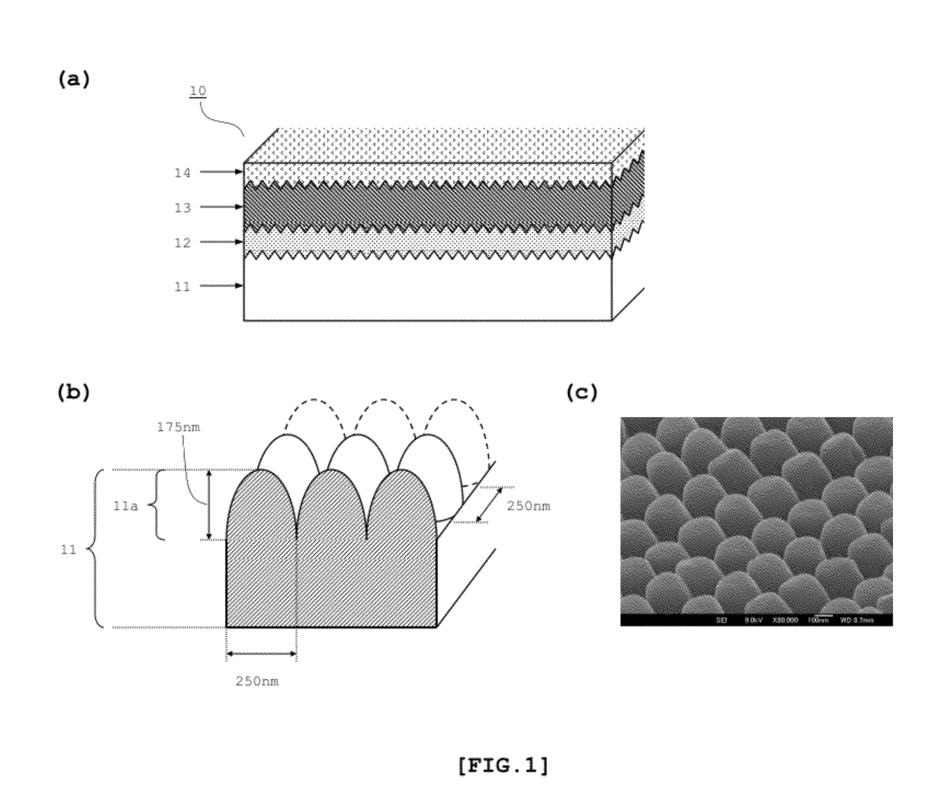

FIG. 1 shows an arrangement of a purification plate 10 in the embodiment. FIG. 1(a) is a diagram showing a laminate structure of the purification plate 10, FIG. 1(b) is a diagram showing a concave-convex structure 11a of a substrate 11, and FIG. 1(c) is a diagram showing a secondary electrophotographic image of the concave-convex structure 11a.

Referring to FIG. 1(a), the purification plate 10 has the substrate 11, a transmissive film 12, a photocatalyst film 13, and an adsorption film 14.

The substrate 11 is made of a light-transmissive material such as polycarbonate, and the refractive index of the substrate is set to 1.6. As shown in FIGS. 1(b), 1(c), the concave-convex structure 11a is formed on a transmissive film 12 side surface of the substrate 11 in such a manner that columnar-shaped protrusions are uniformly arranged at a constant pitch in a matrix. The pitch (width of the columnar-shaped protrusion) of the concave-convex structure 11a is 250 nm in length and breadth, and the height of the columnar-shaped protrusion is 175 nm.

The photographic image shown in FIG. 1(c) is obtained by forming an alloy film of 20 nm on the concave-convex structure 11a by sputtering, followed by image capturing in a state that Pt—Pd is vapor-deposited by 10A for electrophotography.

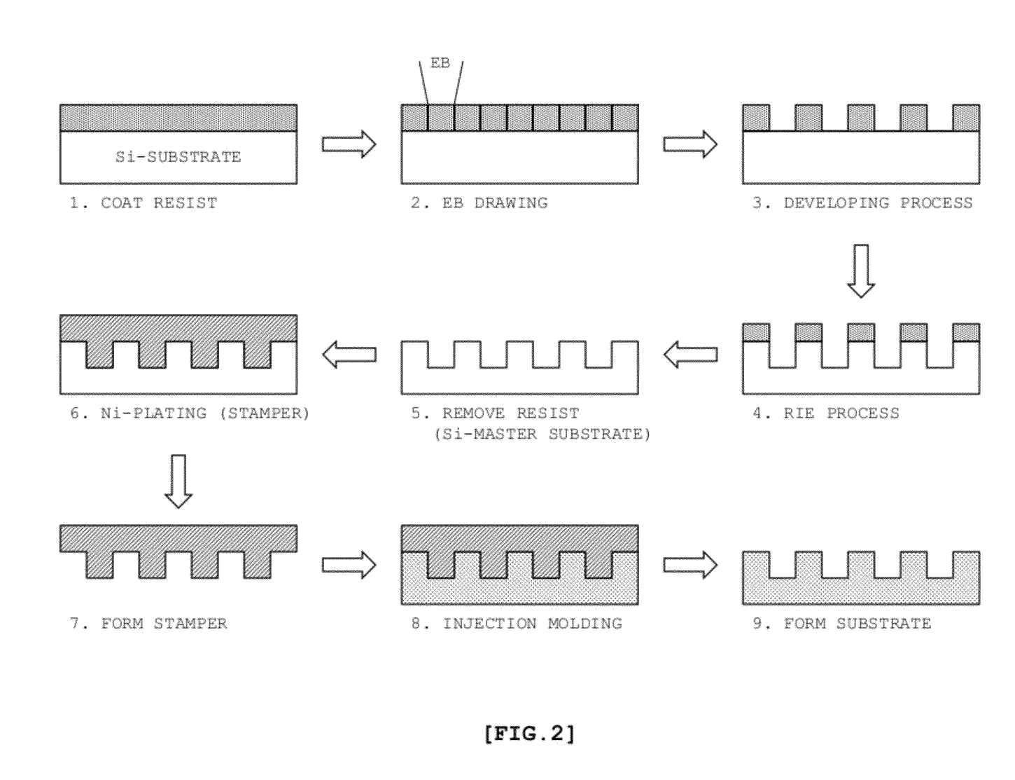

In the following, a sequence of forming the substrate 11 is described referring to FIG. 2.

First, a resist is coated on a silicon master by spin-coating (step 1). Then, a concave-convex structure having the above pitch is formed by EB drawing (electron-beam cutting) (step 2). After the drawing, a developing process is performed (step 3), and then, an RIE process is performed (step 4). Further, the remaining resist is removed by oxygen-plasma asking (step 5). By performing the above steps, a concave-convex structure is formed on the silicon master (Si-master substrate).

Subsequently, the Si master substrate undergoes Ni-plating (step 6) for depositing Ni. Then, a stamper is fabricated by peeling off the deposited Ni-layer from the Si master substrate (step 7). Then, the stamper is subjected to an injection molding process (step 8) for fabricating the substrate 11 (step 9). By performing the above steps, the substrate 11 having a concave-convex structure transferred thereon is formed.

In this embodiment, a light transmissive material such as polyolefin may be used as the material for the substrate 11, other than polycarbonate. Further alternatively, a biodegradable material such as polylactate may be used, other than the above. Use of a biodegradable material is advantageous in reducing an environmental load or the like at the time of disposal.

Alternatively, laser beam cutting may be applied, in place of EB drawing. In this case, a photoresist layer is coated on the silicon master. Further, laser light of about 400 nm in wavelength is used as a cutting beam.

Referring back to FIG. 1(a), the transmissive film 12 is laminated on the concave-convex structure 11a of the substrate 11 formed by the aforementioned sequence by a sputtering method. The transmissive film 12 is made of Al₂O₃, and the refractive index of the transmissive film 12 is set to 1.6 so that the refractive indexes of the transmissive film 12 and the substrate 11 are substantially equal to each other. Further, the upper surface and the lower surface of the transmissive film 12 are formed into a concave-convex structure reflecting the concave-convex structure 11a of the substrate 11. Since the transmissive film 12 is made of a non-electrolytic inorganic material, the transmissive film 12 is free from corrosion by a photocatalytic reaction on the photocatalyst film 13 to be described later.

Further, the film thickness and the Ra (surface roughness) of the transmissive film 12 are set to such values that the substrate 11 is not corroded by the photocatalyst film 13, and that light to be entered from the substrate 11 side is sufficiently allowed to impinge on the photocatalyst film 13.

Specifically, if the film thickness of the transmissive film 12 is set to a small value, a hole may be formed in the transmissive film 12 depending on the setting of Ra, and the substrate 11 and the photocatalyst film 13 may be contacted with each other through the hole. If the substrate 11 is contacted with the photocatalyst film 13 as described above, the substrate 11 may be corroded by the photocatalytic reaction on the photocatalyst film 13 to be described later, and the substrate 11 maybe degraded. Further, if the Ra of the transmissive film 12 is set to a large value, a hole may be formed in the transmissive film 12 depending on the film thickness of the transmissive film 12. In this case, the substrate 11 may also be corroded by the photocatalytic reaction on the photocatalyst film 13, and the substrate 11 may be degraded. On the other hand, if the film thickness of the transmissive film 12 is set to a large value for preventing formation of a hole, light to be entered from the substrate 11 side may be absorbed by the transmissive film 12, which makes it difficult to allow the light to impinge on the photocatalyst film 13. As a result, the progress of photocatalytic reaction on the photocatalyst film 13 may be obstructed.

In view of the above, it is necessary to properly set the film thickness and the Ra of the transmissive film 12. The control on the Ra of the transmissive film 12 is performed by adjusting the gas pressure at the time of sputtering.

In an experiment conducted by the inventor of the present application, the substrate 11 was corroded by the photocatalytic reaction on the photocatalyst film 13 in the case where the film thickness of the transmissive film 12 was set to 7 nm, and the Ra of the transmissive film 12 was set to 0.8 nm, but the substrate 11 was not corroded by the photocatalytic reaction on the photocatalyst film 13 in the case where the film thickness of the transmissive film 12 was set to 7 nm, and the Ra of the transmissive film 12 was set to 0.6 nm. This shows that it is desirable to form the transmissive film 12 in such a manner that the Ra of the film surface is equal to or less than about 10% of the film thickness. Further, it is desirable to set the film thickness of the transmissive film 12 in the range from about 3 to 80 nm so that light to be entered from the substrate 11 side is sufficiently transmitted toward the photocatalyst film 13. In view of the above, in this embodiment, the film thickness of the transmissive film 12 was set to 7 nm, and the Ra of the transmissive film 12 was set to 0.66. The gas pressure at the time of sputtering was set to 0.3 Pa.

The photocatalyst film 13 is laminated on the upper surface of the transmissive film 12 by a sputtering method. The photocatalyst film 13 is made of TiO₂, and the refractive index of the photocatalyst film 13 is set to 2.5. Further, the upper surface and the lower surface of the photocatalyst film 13 are formed into a concave-convex structure reflecting the concave-convex structure formed on the upper surface of the transmissive film 12. With the above arrangement, a structure reflecting the concave-convex structure 11a on the surface of the substrate 11 is formed on the upper surface (reaction surface) of the photocatalyst film 13. This increases the surface area of the upper surface of the photocatalyst film 13, and makes it easy to cause a photocatalytic reaction.

The surface of the photocatalyst film 13 itself after film formation can be made porous by adjusting the gas pressure in laminating. By performing the above operation, the photocatalyst film 13 itself becomes porous, which makes it possible to increase the surface area of the photocatalyst film 13, and the surface area of the photocatalyst film 13 can be further increased by the concave-convex structure 11a of the substrate 11. If the film thickness of the photocatalyst film 13 is small, it is impossible or difficult to completely cover the upper surface of the transmissive film 12 by the photocatalyst film 13. On the other hand, if the film thickness of the photocatalyst film 13 is large, the concave-convex structure formed on the upper surface of the transmissive film 12 is not reflected on the upper surface (reaction surface) of the photocatalyst film 13, and in addition to the above, absorption of light to be entered from the transmissive film 12 side on the photocatalyst film 13 blocks the light from impinging on the upper surface of the photocatalyst film 13. In view of the above, in this embodiment, the film thickness of the photocatalyst film 13 was set to 80 nm so that the upper surface of the transmissive film 12 was sufficiently covered, and a sufficient amount of light impinging on the upper surface of the transmissive film 12 was obtained.

TiO₂ forming the photocatalyst film 13 contains anatase crystal particles. Anatase crystal absorbs ultraviolet light of 388 nm or less in wavelength from a band gap material, and causes a photocatalytic reaction. Further, since anatase crystal exists in the photocatalyst film 13 in the form of particles, the anatase crystal is uniformly distributed in the substrate 11, no matter how intricate the shape of the substrate 11 is. This makes it easy to cause a photocatalytic reaction in a wide range over the photocatalyst film 13 with enhanced efficiency.

Further, it is known that TiO₂ forms a rutile structure, an amorphous structure, a brookite structure, other than the anatase crystal structure, and the photocatalytic reaction differs depending on the structure. Specifically, the reaction activity and the reaction wavelength differ in each of the structures. TiO₂ forming the photocatalyst film 13 used in this embodiment contains plural types of the structures. Specifically, TiO₂ forming the photocatalyst film 13 used in this embodiment contains amorphous matter, defects in crystal anatase structure, and particles including a trace of nitrogen contained at the time of sputtering, rutile particles. By the containment of these matters, the photocatalytic reaction on the photocatalyst film 13 progresses also by the light having a wavelength in a visible light region in the range from 400 to 500 nm. Thus, in the case where an LED or a semiconductor laser is used as a light source for causing a photocatalytic reaction, light use efficiency is enhanced, even in the case where the light to be emitted from these light sources contains visible light depending on the temperature or a difference in the type of the light source. A structure to be formed by TiO₂ may not necessarily include all the aforementioned structures, other than anatase crystal structure, as far as TiO₂ contains particles that are activated by visible light.

The photocatalyst film 13 photocatalytically acts on a material adhered to the photocatalyst film 13. Examples of a material which undergoes a photocatalytic action include ammonium, acetaldehyde, hydrogen sulfide, methyl mercaptan, formaldehyde, acetic acid, toluene, bacteria, and oils. These materials undergo a photocatalytic action and are decomposed into carbon dioxide, water, and the like.

The adsorption film 14 is laminated on the upper surface of the photocatalyst film 13 by a sputtering method. The adsorption film 14 is composed of SiO₂. SiO₂ has moisture absorbency, and has a property that it is likely to bind with water molecules in the air or a vapor phase gas. With use of the adsorption film 14 having the above property, the material in the air that exists on the upper surface of the adsorption film 14 is easily adhered to the adsorption film 14. Further, the material adsorbed onto the adsorption film 14 is trapped on the adsorption film 14 and undergoes a photocatalytic action.

The adsorption film 14 is laminated on the photocatalyst film 13 in such a manner that the upper surface of the photocatalyst film 13 is not entirely coated. Specifically, multitudes of micropores are formed in the adsorption film 14 by lowering the gas pressure at the time of sputtering (specifically, 0.8 through 1 Pa or higher), or increasing the sputtering rate (70 Å/min or larger). By performing the above operation, the material adhered to the upper surface of the adsorption film 14 is contacted with the photocatalyst film 13 through the micropores. It is desirable to set the film thickness of the adsorption film 14 to such a value that the material adhered to the adsorption film 14 is efficiently contacted with the photocatalyst film 13, specifically, desirable to set the film thickness in the range from about 3 to 100 nm. In this embodiment, the film thickness of the adsorption film 14 is set to 7 nm.

With use of the purification plate 10 as described above, in the case where ultraviolet light of 375 nm in wavelength is irradiated toward the purification plate 10 from a surface (hereinafter, called as an “incident surface”) of the substrate 11 opposite to the surface thereof facing the transmissive film 12, the ultraviolet light is transmitted through the substrate 11, the transmissive film 12, the photocatalyst film 13, and is allowed to impinge on the upper surface (hereinafter, called as a “reaction surface”) of the photocatalyst film 13. By performing the above operation, the material adhered to the adsorption film 14 and in contact with the reaction surface is operable to undergo a photocatalytic action.

In the above arrangement, since the transmissive film 12 is disposed between the substrate 11 and the photocatalyst film 13, it is possible to suppress corrosion of the substrate 11 by the photocatalytic action on the photocatalyst film 13. Further, since a concave-convex structure is formed on the reaction surface, the surface area of the reaction surface to be contacted with the material is increased, which makes it easy to cause a photocatalytic reaction on the photocatalyst film 13. Further, ultraviolet light causes multiple reflections on the surface of the concave-convex structure formed on the reaction surface. This increases the number of times of reaction between the photocatalyst film 13 and ultraviolet light, and makes it easy to cause a photocatalytic reaction on the photocatalyst film 13.

Further, with use of the purification plate 10, a material is easily trapped on the adsorption film 14. This is advantageous in carrying out a photocatalytic reaction with the material with enhanced efficiency, as compared with the case where the adsorption film 14 is not provided.

Further, with use of the purification plate 10, a fine concave-convex structure is formed on facing surfaces between the substrate 11 and the transmissive film 12, and facing surfaces between the transmissive film 12 and the photocatalyst film 13; and the pitch (250 nm) of the concave-convex structure is set to a value smaller than the wavelength (375 nm) of ultraviolet light for a photocatalytic reaction. This suppresses reflection of ultraviolet light to be entered from the incident surface on the interface between the substrate 11 and the transmissive film 12, the facing surface of the transmissive film 12 and the interface of the photocatalyst film 13. Further, since the refractive indexes of the substrate 11 and the transmissive film 12 are substantially equal to each other, it is possible to further suppress reflection of light to be entered from the incident surface on the interface between the transmissive film 12 and the substrate 11. By performing the above operation, light to be entered from the incident surface of the purification plate 10 is allowed to impinge on the photocatalyst film 13 with enhanced efficiency. The transmittance of the purification plate 10 fabricated as described above is 70% or larger at the wavelength λ=375 nm.

In this embodiment, the refractive indexes of the transmissive film 12 and the substrate 11 are set to 1.6. However, in the case where the refractive indexes of the transmissive film 12 and the substrate 11 are set to a value other than the above, materials for the transmissive film 12 and the substrate 11 may be optionally selected depending on the set refractive indexes. It is desirable to make the refractive indexes of the transmissive film 12 and the substrate 11 equal to each other. However, even in the case where the refractive indexes of the transmissive film 12 and the substrate 11 differ from each other, it is desirable to select materials for the transmissive film 12 and the substrate 11 in such a manner that the refractive indexes of the transmissive film 12 and the substrate 11 are approximated to each other.

Further, with use of the purification plate 10, it is possible to cause a photocatalytic reaction on the reaction surface by allowing light to be entered from the incident surface. Accordingly, even in the case where a large amount of a material to be purified is adhered to the adsorption film 14, it is possible to cause a photocatalytic reaction on the reaction surface without stagnation.

Alternatively, the transmissive film 12, the photocatalyst film 13, the adsorption film 14 may be disposed on the lower surface side of the substrate 11, as well as on the upper surface side of the substrate 11, in the same manner as on the upper surface side of the substrate 11. In the modification, the adsorption film 14 is constituted of a light-transmissive film. In the modification, when light is entered from the lower surface side, a photocatalytic reaction occurs both on the lower surface (reaction surface) of the lower surface side transmissive film 12 and on the upper surface (reaction surface) of the upper surface side transmissive film 12, with use of one light source. In the modification, however, it is necessary to take into account a likelihood that a large amount of a material may be adsorbed onto the lower surface side surface, which may lower the light transmittance.

Further, the material to be used for each of the layers of the purification plate 10 is not limited to the above. Furthermore, the thickness of each of the layers maybe changed as the material changes, as necessary. In addition, the wavelength of light to be used for a photocatalytic reaction may be changed depending on the material to be used for the photocatalyst film 13, as necessary. In the case where the wavelength of light to be used for a photocatalytic reaction is changed as described above, it is desirable to adjust the pitch of the concave-convex structure in accordance with the change in the wavelength. Specifically, it is necessary to set the pitch of the concave-convex structure to a value smaller than the wavelength of light to be used for a photocatalytic reaction in order to suppress reflection of the light on the interface between the substrate 11 and the transmissive film 12, the facing surface of the transmissive film 12 and the interface of the photocatalyst film 13 in the same manner as described above.

In this embodiment, the description has been made based on the premise that light is irradiated from the substrate 11 side. However, the photocatalytic reaction surface area drastically increases by formation of the concave-convex structure and the porous structure of TiO₂. Accordingly, as far as the amount of a target material adhered to the adsorption film 14 is not so large, irradiation of light from the adsorption film 14 side is operable to enhance the purification performance, as compared with a conventional purification plate devoid of these structures. However, if the amount of a target material adhered to the adsorption film 14 is intolerably large, and the light from the adsorption film 14 side does not impinge on the reaction surface, the result to be obtained by using the purification plate 10 may be substantially the same as the result to be obtained by using the conventional purification plate. In view of the above, in this embodiment, it is desirable to allow light to be entered from the substrate 11 side.

<Measurement Example>

The inventor conducted measurement on the reflectance lowering action by the concave-convex structure, and the following is a description about the measurement.

In the measurement, as shown in FIG. 1(c), a concave-convex structure was formed on a substrate in such a manner that columnar-shaped protrusions were uniformly arranged at a constant pitch in a matrix. The pitch (distance between adjacent columnar-shaped protrusions) of the concave-convex structure was 250 nm in length and breadth, and the height of the columnar-shaped protrusion was 170 nm. The concave-convex structure was formed by the steps shown in FIG. 2. Specifically, a substrate having a concave-convex structure transferred thereon was formed by the steps shown in FIG. 2. In the measurement, polycarbonate was used as a substrate material.

In the measurement, a Co50Al50at.% alloy film (reflection film) of 20 nm was formed on the substrate formed as described above by sputtering. In the measurement, only the alloy film (reflection film) was formed on the substrate.

The alloy film (reflection film) was formed as follows. Vacuum drawing was carried out up to the pressure of 5×10⁻⁵ Pa or lower in a vacuum chamber. Thereafter, Ar gas was introduced into the chamber, and sputtering was carried out in the atmosphere of 0.6 Pa. In the chamber, a Co target and an Al target were placed, and a Co50Al 50at.% alloy film (reflection film) was formed by a Co sputtering method i.e. an alloying method by supplying an electric power to each of the targets at the same time. The substrate was allowed to revolve at 40 rpm during discharging for uniformly forming the alloy film (reflection film).

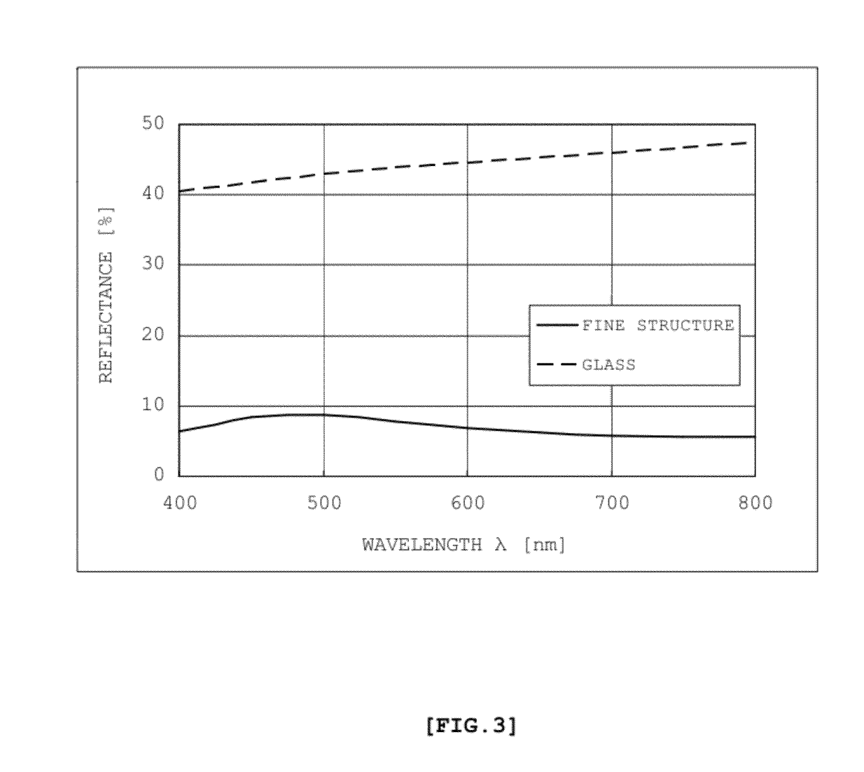

After the alloy film (reflection film) was formed, reflectance measurement was carried out. FIG. 3 shows a result on the reflectance measurement. FIG. 3 also shows, as a comparative example, a measurement result on the reflectance of a structural member obtained by forming a Co50Al50at.% alloy film of 20 nm on a glass substrate having a flat reflection film surface by sputtering. Light was irradiated from the alloy film (reflection film) side in such a manner that plural concave-convex structures were included in an irradiation area.

FIG. 3 shows that the reflectance is lowered by about 35 to 40% in the case where the aforementioned concave-convex structure was formed on a substrate, as compared with the case where a glass substrate has a flat Al—Co film. This shows that forming a concave-convex structure enables to make the reflectance difference large with respect to a specular surface portion devoid of the concave-convex structure.

FIG. 3 shows that the aforementioned reflectance lowering action is obtained in the case where at least the pitch (250 nm) of the concave-convex structure is smaller than the wavelength (375 nm) of light to be used for a photocatalytic reaction. Accordingly, with use of the purification plate 10, setting the pitch of the concave-convex structure smaller than the wavelength of light to be used for a photocatalytic reaction enables to suppress reflection of the light on the interface between the substrate 11 and the transmissive film 12, the facing surface of the transmissive film 12 and the interface of the photocatalyst film 13.

<Deodorizing Device>

The following is an example, in which the purification plate 10 is applied to a deodorizing device.

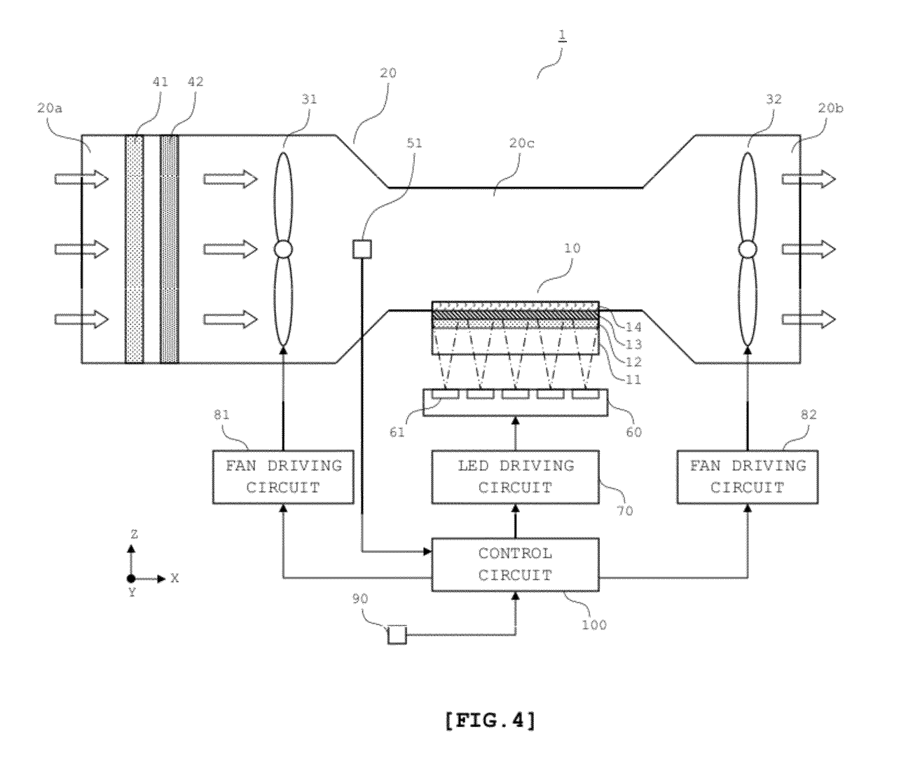

FIG. 4 is a diagram showing an arrangement of a deodorizing device 1.

The deodorizing device 1 is provided with a purification plate 10, an air passage 20, fans 31, 32, filters 41, 42, an odor sensor 51, a light emission unit 60, an LED driving circuit 70, fan driving circuits 81, 82, a power source switch 90, and a control circuit 100.

The air passage 20 is formed of a hollow tubular member, and is configured in such a manner that the air is allowed to flow in X-axis direction through the air passage 20. An air intake port 20a and an air exhaust port 20b are respectively formed at an entrance and an exit of the air passage 20. Further, a purification region 20c in which the purification plate 10 is disposed is formed near the center of the air passage 20.

As described above, the purification plate 10 is configured in such a manner that the substrate 11, the transmissive film 12, the photocatalyst film 13, the adsorption film 14 are laminated one over the other in parallel to X-Y plane. Further, the purification plate 10 is disposed in such a manner as to protrude in the purification region 20c.

The fans 31, 32 cause the air to flow from the air intake port 20a toward the air exhaust port 20b. With the above arrangement, the air in the vicinity of the air intake port 20a is drawn in through the air intake port 20a by the fan 31, passes through the purification region 20c, and is drawn out through the air exhaust port 20b by the fan 32.

The filter 41 removes large dust particles contained in the air that is drawn in through the air intake port 20a, and the filter 42 removes small dust particles contained in the air that is drawn out from the filter 41 side. The odor sensor 51 detects an odor component contained in the air that is drawn out toward the purification region 20c by the fan 31. A detection signal from the odor sensor 51 is outputted to the control circuit 100.

The light emission unit 60 has a plurality of LEDs 61 on X-Y plane. These LEDs 61 emit light of 375 nm in wavelength toward the incident surface of the purification plate 10. The LED driving circuit 70 drives the LEDs 61 disposed in the light emission unit 60 in accordance with an instruction from the control circuit 100.

The fan driving circuits 81, 82 respectively drive the fans 31, 32 in accordance with an instruction from the control circuit 100. The rotation number of the fans 31, 32 is controlled by the control circuit 100. The power source switch 90 is provided with a switch for switching between ON and OFF states, and the operator is allowed to switch the power source of the deodorizing device 1 between ON and OFF states by switching the switch. A switching signal of the power source switch 90 is outputted to the control circuit 100. The control circuit 100 controls the LED driving circuit 70, and the fan driving circuits 81, 82, based on an output signal from the odor sensor 51 and the switching signal from the power source switch 90.

In the deodorizing device 1 having the above arrangement, dusts in the air drawn in through the air intake port 20a are removed by the filters 41, 42 by driving the fan 31, and then, the air after the dust removal is drawn to the purification region 20c. When the air drawn to the purification region 20c is contacted with the adsorption film 14 of the purification plate 10, the material contained in the air is trapped on the adsorption film 14, and is contacted with the reaction surface of the photocatalyst film 13. In this state, when light is irradiated from the LEDs 61 of the light emission unit 60 onto the photocatalyst film 13, a photocatalytic reaction occurs on the reaction surface, and the material in contact with the reaction surface is decomposed. The material that has been decomposed by the action of the purification plate 10 is drawn from the purification region 20c toward the air exhaust port 20b by driving the fans 31, 32, whereby the air is drawn out through the air exhaust port 20b. In this way, the material contained in the air in the vicinity of the deodorizing device 1 is purified. In the arrangement of this embodiment, since the air passage and the driving circuit for e.g. the LEDs 61 can be disposed at such positions that the purification plate 10 is interposed therebetween, it is possible to purify even if a material to be purified is a gas that may corrode a circuit.

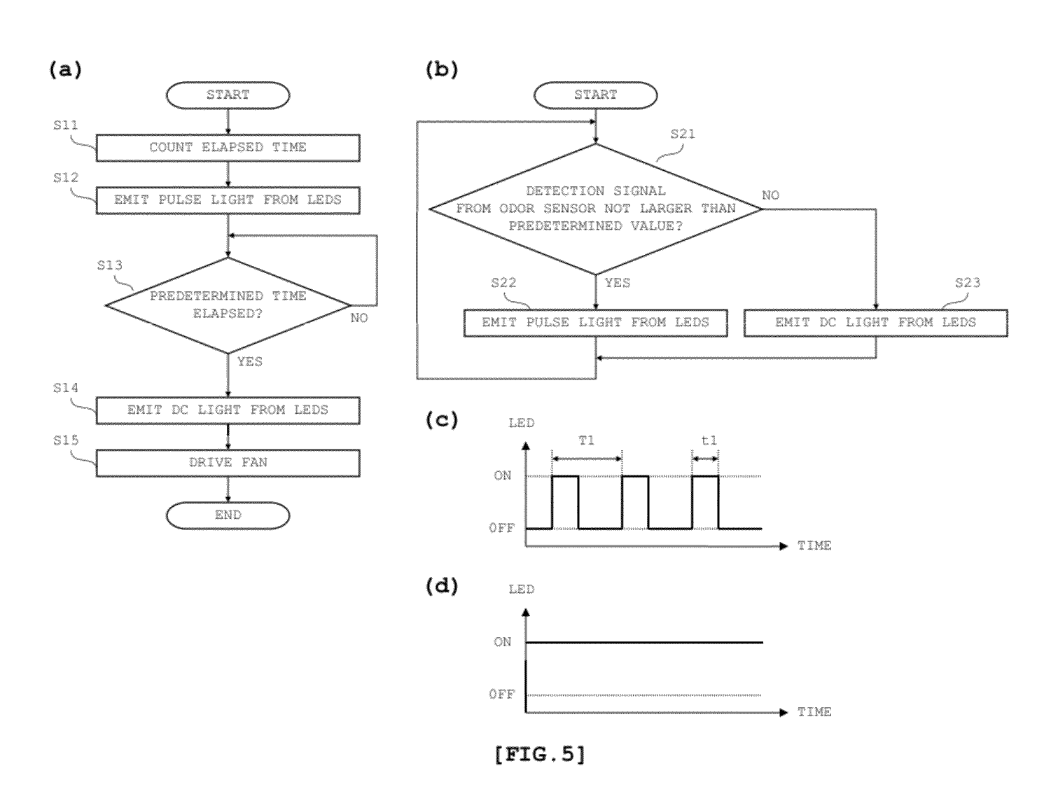

FIG. 5 is a diagram for describing a control to be performed by the control circuit 100.

FIG. 5(a) is a flowchart showing a control of the LEDs 61 and the fans 31, 32 to be performed immediately after the power source switch 90 of the deodorizing device 1 is switched from an OFF-state to an ON-state.

When the power source of the deodorizing device 1 is turned on, first, a lapse of time from the time when the power source of the deodorizing device 1 has been turned on is counted (S11). Subsequently, the control circuit 100 causes the LEDs 61 to emit pulse light via the LED driving circuit 70 (S12). In this example, as shown in FIG. 5(c), when the LEDs 61 emit pulse light, the LEDs 61 are kept in an ON-state (turned on) for a time t1 at every cycle T1.

Referring back to FIG. 5(a), in the case where the control circuit 100 determines that the lapse of time from the time when the power source of the deodorizing device 1 has been turned on reaches a predetermined time (S13: YES), the processing is proceeded to Step S14; and in the case where the control circuit 100 determines that the lapse of time does not reach the predetermined time (S14: NO), the processing is waited.

In Step S14, the control circuit 100 causes the LEDs 61 to emit DC light via the LED driving circuit 70. In this example, as shown in FIG. 5(d), when the LEDs 61 are caused to emit DC light, the LEDs 61 are constantly brought to an ON-state (turned on). Referring back to FIG. 5(a), in Step S15, the control circuit 100 drives the fans 31, 32 via the fan driving circuits 81, 82. In this way, the deodorizing device 1 is ordinarily operated.

When the LEDs 61 and the fans 31, 32 are controlled as described above, it is possible to suppress drawing out, through the air exhaust port 20b, of an un-purified material adhered to the adsorption film 14 at the time when the power source of the deodorizing device 1 is turned off, immediately after the power source of the deodorizing device 1 is turned on.

Specifically, since the adsorption film 14 has high adsorbability, the material floating in the vicinity of the purification region 20c at the time when the power source of the deodorizing device 1 is turned off is adhered to the adsorption film 14. If the power source of the deodorizing device 1 is turned on and the LEDs 61 are caused to emit DC light in this state, the photocatalyst film 13 absorbs the light energy, and the temperature of the photocatalyst film 13 instantaneously rises. As a result, the un-purified material adhered to the adsorption film 14 may be released in the air around the purification region 20c without being purified. If the fans 31, 32 are driven in this state, the un-purified material released in the air around the purification region 20c may be drawn out through the air exhaust port 20b. In addition to the above, the un-purified material may leave the adsorption film 14 by the airstream generated by the fans 31, 32, and may be drawn out through the air exhaust port 20b.

On the other hand, in the control shown in FIG. 5(a), as described above, since the LEDs 61 are caused to emit pulse light during a predetermined period from the point of time when the power source of the deodorizing device 1 is turned on, the temperature rise of the photocatalyst film 13 in this period is moderate. As a result, the un-purified material adhered to the adsorption film 14 is trapped on the adsorption film 14, and purified. By performing the above operation, it is possible to suppress releasing the un-purified material into the air around the purification region 20c. Further, since the fans 31, 32 are stopped when the above operation is performed, it is possible to suppress the un-purified material adhered to the adsorption film 14 from leaving the adsorption film 14 by the airstream, and from drawing out through the air exhaust port 20b. In this example, the fans 31, 32 are stopped. Alternatively, it may be possible to drive (moderately drive) the fans 31, 32 at such a low rotation number that the un-purified material adhered to the adsorption film 14 is not released into the air.

The predetermined time used in the determination in Step S13 is set to a time required for sufficiently reducing the un-purified material adhered to the adsorption film 14, in other words, a time required for purifying the adsorption film 14 to such an extent as to allow subsequent adsorption. In this way, after the predetermined time has elapsed, the un-purified material adhered to the adsorption film 14 is reduced, and the material floating in the purification region 20c is allowed to be adsorbed onto the adsorption film 14. Thus, it is possible to quickly start drawing in the air around the deodorizing device 1.

FIG. 5(b) is a flowchart showing a control of the LEDs 61 when the deodorizing device 1 is ordinarily operated.

In the case where it is determined that a detection signal from the odor sensor 51 is equal to or smaller than a predetermined value (S21: YES), the control circuit 100 causes the LEDs 61 to emit pulse light (S22); and in the case where it is determined that a detection signal from the odor sensor 51 is larger than the predetermined value (S21: NO), the control circuit 100 causes the LEDs 61 to emit DC light (S23).

Controlling the LEDs 61 as described above enables to securely purify the material to be purified, even in the case where only a trace of the material to be purified exists in the air that is drawn out to the purification region 20c when the deodorizing device 1 is ordinarily operated. Specifically, if the LEDs 61 are caused to emit DC light in a condition that only a trace of the material to be purified exists in the air, the temperature of the photocatalyst film 13 rises. As a result, the temperature of the air around the purification plate 10 also rises. This makes it difficult to allow the material to be purified that is contained in the air to adhere to the adsorption film 14, and makes it difficult to purify the material to be purified. However, causing the LEDs 61 to emit pulse light in the above condition suppresses the temperature rise of the photocatalyst film 13, thereby making it easy to allow the material to be purified to adhere to the adsorption film 14, and purify the material to be purified.

As described above, in the deodorizing device 1, the material contained in the air that is drawn in through the air intake port 20a is purified by the photocatalytic action on the photocatalyst film 13 of the purification plate 10, and is drawn out through the air exhaust port 20b. This enables to purify the air in the vicinity of the deodorizing device 1.

Further, in the deodorizing device 1, it is possible to suppress drawing out, through the air exhaust port 20b, of the un-purified material adhered to the adsorption film 14 at the point of time when the power source of the deodorizing device 1 is turned off, even immediately after the power source of the deodorizing device 1 is turned on. This enables to suppress odor release to the outside of the deodorizing device 1, immediately after the power source of the deodorizing device 1 is turned on. Further, in the case where the amount of the material to be purified is very small when the deodorizing device 1 is ordinarily operated, the LEDs 61 are caused to emit pulse light, which suppresses the temperature rise of the photocatalyst film 13. This enables to efficiently adsorb the trace of the material to be purified onto the purification plate 10, and securely purify the material to be purified.

<Modification of Deodorizing Device>

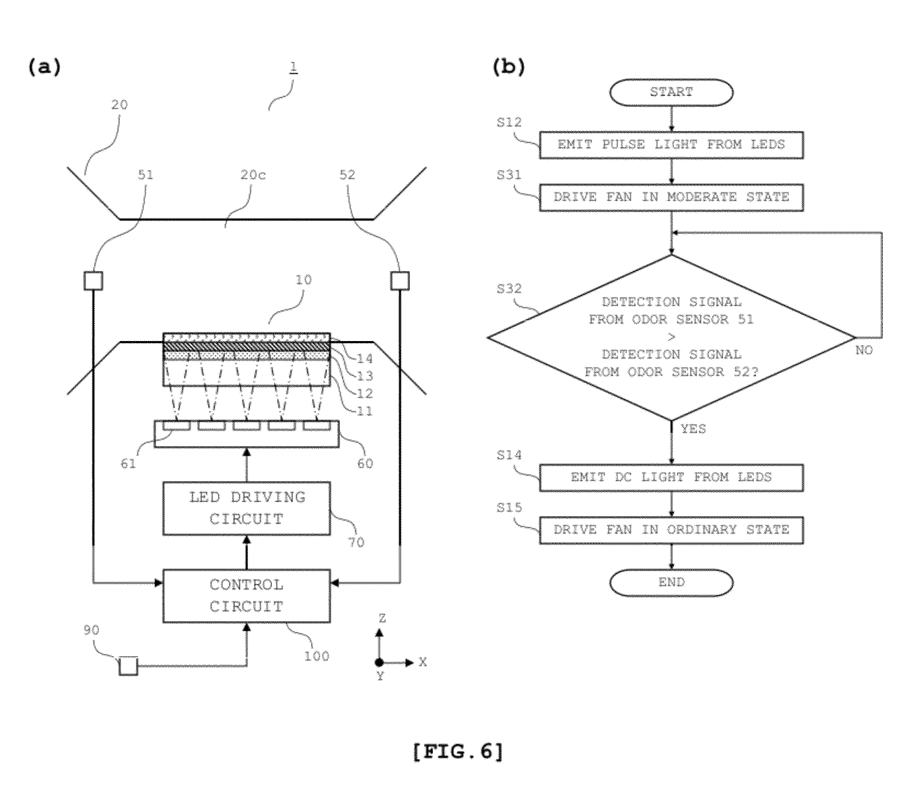

FIG. 6(a) is a diagram showing an arrangement of the deodorizing device 1 in the modification.

The deodorizing device 1 in the modification is configured in such a manner that the deodorizing device 1 shown in FIG. 4 is additionally provided with an odor sensor 52. The odor sensor 52 has substantially the same arrangement as the odor sensor 51, and detects an odor component contained in the air that is drawn out from the purification region 20c toward the fan 32. A detection signal from the odor sensor 52 is outputted to the control circuit 100. In FIG. 6(a), only the purification region 20c and the vicinity thereof are illustrated for simplifying the description.

FIG. 6(b) is a flowchart showing a control of the LEDs 61 and the fans 31, 32 immediately after the power source of the deodorizing device 1 is turned on. In the modification, Steps S11, S13 are deleted, and Steps S31, S32 are added to the flowchart shown in FIG. 5(a).

When the power source of the deodorizing device 1 is turned on, the control circuit 100 causes the LEDs 61 to emit pulse light via the LED driving circuit 70 (S12) for driving (moderately driving) the fans 31, 32 at a rotation number lower than that in an ordinary state by several stages.

Subsequently, the control circuit 100 determines whether the detection signal from the odor sensor 51 is larger than the detection signal from the odor sensor 52 (S32). If it is determined that the detection signal from the odor sensor 51 is larger than the detection signal from the odor sensor 52 (S32: YES), the processing is proceeded to Steps S14 and S15 for driving the LEDs 61 and the fans 31, 32 in an ordinary state. On the other hand, if it is determined that the detection signal of the odor sensor 51 is equal to or smaller than the detection signal of the odor sensor 52 (S32: NO), the processing is waited.

In this example, if the detection signal from the odor sensor 52 disposed at the downstream side is smaller than the detection signal from the odor sensor 51 disposed at the upstream side (S32: YES), the control circuit 100 determines that the un-purified material adhered to the adsorption film 14 at the point of time when the power source of the deodorizing device 1 has been turned off is substantially purified, and causes the LEDS 61 and the fans 31, 32 to drive in an ordinary state (S14, S15).

Controlling the LEDs 61 and the fans 31, 32 as described above enables to suppress drawing out, through the air exhaust port 20b, of the un-purified material after the power source of the deodorizing device 1 is turned on also in the modification.

<Modification on Arranged Position of Purification Plate>

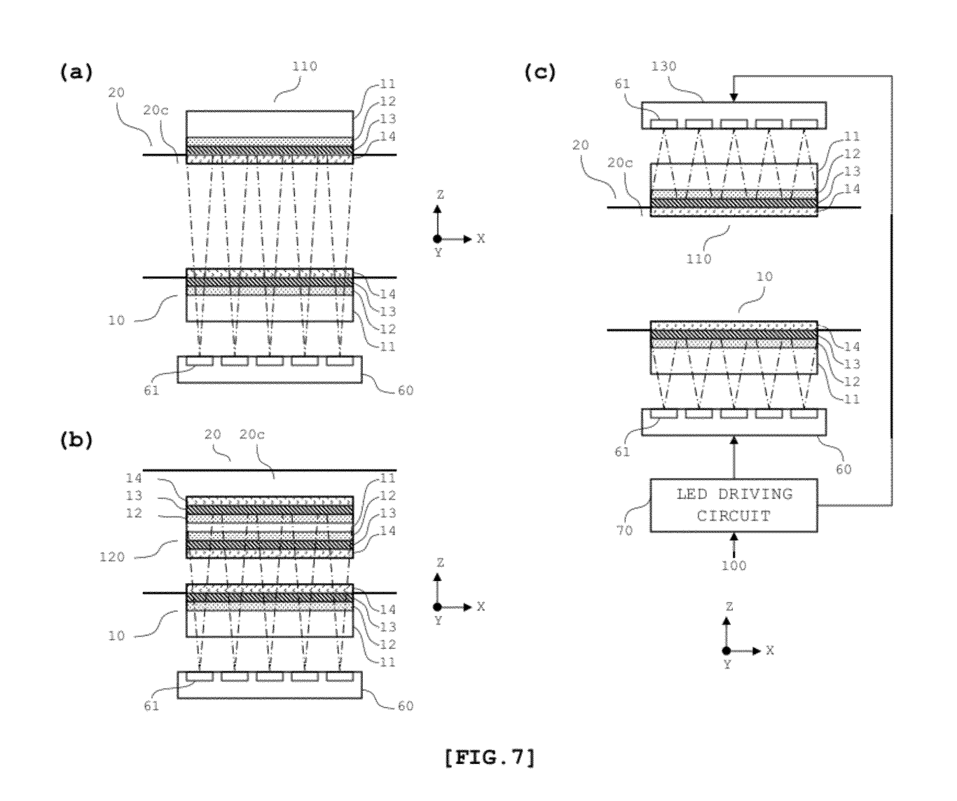

The arranged position of the purification plate 10 is not limited to the arranged position shown in FIG. 4 and FIG. 6(a), and another purification plate substantially the same as the purification plate 10 may be disposed in the following manner. In FIG. 7, to simplify the description, only a purification region 20c and the vicinity thereof are illustrated.

In a deodorizing device 1 shown in FIG. 7(a), a purification plate 110 is disposed to be symmetrical to the purification plate 10 shown in FIG. 4 and FIG. 6(a) with respect to the purification region 20c.

In the modification, light to be emitted from the LEDs 61 is absorbed by the purification plate 10. The light transmitted through the purification plate 10 is further absorbed by an adsorption film 14 of the purification plate 110 and by the material adhered to the adsorption film 14 of the purification plate 110. However, a part of the light emitted from the LEDs 61 impinges on a photocatalyst film 13 of the purification plate 110 to cause a photocatalytic reaction on the photocatalyst film 13 of the purification plate 110. Performing the above operation is more advantageous in purifying the air in the purification region 20c, as compared with the deodorizing device 1 shown in FIG. 4 and FIG. 6(a). In FIG. 7(a), another purification plate 110 may be disposed on an inner wall surface of the purification region 20c, at a position between the purification plate 10 and the purification plate 110.

In a deodorizing device 1 shown in FIG. 7(b), another purification plate 120 is disposed in Z-axis plus direction of the purification plate 10 shown in FIG. 4 and FIG. 6(a). A transmissive film 12, a photocatalyst film 13, an adsorption film 14 are disposed in such a manner that a photocatalytic action is performed on both surfaces of the purification plate 120 in Z-axis plus direction and Z-axis minus direction. Further, as shown in FIG. 7(b), the purification plate 120 is fixedly mounted on the inner wall surface of the purification region 20c.

In the above modification, light to be emitted from the LEDs 61 is transmitted through the purification plate 10, and impinges on both of the photocatalyst films 13 disposed on both sides of the purification plate 120 corresponding to Z-axis plus direction and Z-axis minus direction for causing a photocatalytic reaction on each of the photocatalyst films 13. The above arrangement is more advantageous in purifying the air in the purification region 20c by remarkably increasing the reaction surface area with use of one light source, as compared with the embodiment of the deodorizing device. In FIG. 7(b), another purification plate substantially having the same arrangement as the purification plate 120 may be disposed in the purification region 20c in Z-axis plus direction of the purification plate 120.

In a deodorizing device 1 shown in FIG. 7(c), a purification plate 110 and a light emission unit 130 are disposed to be symmetrical to the purification plate 10 and the light emission unit 60 shown in FIG. 4 and FIG. 6(a) with respect to the purification region 20c. The light emission unit 130 has substantially the same arrangement as the light emission unit 60, and light emitted from LEDs 61 disposed in the light emission unit 130 is irradiated onto a photocatalyst film 13 of the purification plate 110. Light from the light emission unit 60 impinges on the purification plate 110 through the purification plate 10. Further, light from the light emission unit 130 impinges on the purification plate 10 through the purification plate 110. By performing the above operation, the amount of light to be irradiated onto the reaction surfaces of the photocatalyst films 13 of the purification plates 10, 110 remarkably increases, and the photocatalytic reaction is more activated.

In the above modification, a photocatalytic reaction occurs on the purification plate 110, as well as on the purification plate 10. Accordingly, the modification is more advantageous in purifying the air in the purification region 20c, as compared with the embodiment of the deodorizing device. In FIG. 7(c), a purification plate and a light emission unit are disposed as opposed to each other in Z-axis direction. Alternatively, a purification plate and a light emission unit may be disposed as opposed to each other in Y-axis direction. The modification is more advantageous in purifying the air in the purification region 20c. Further, in the arrangement shown in FIG. 7(c), the purification plate 120 shown in FIG. 7(b) may be disposed between the purification plate 10 and the purification plate 110. The modification is advantageous in efficiently causing a photocatalytic reaction on the upper surface and the lower surface of the purification plate 120, because light transmitted through the purification plates 10, 110 is irradiated onto the purification plate 120 from above and below the purification plate 120.

As described above, disposing purification plates in multiple stages in the light emission direction with respect to a light emission unit is advantageous in minimizing the number of the light emission units and lowering the cost. Further, since the light transmitted through the purification plates can be re-used, the light use efficiency is enhanced. Furthermore, it is possible to cause a photocatalytic reaction on a large area within a limited space.

In the case where purification plates are provided in multiple stages, if the transmittance of the purification plates is lowered, light may not impinge on the purification plates facing the light, which may make it difficult to obtain the above effect. In view of the above, in this embodiment, the purification plate 10 was configured in such a manner that the transmittance of the purification plate 10 including the substrate 11, the transmissive film 12, the photocatalyst film 13, the adsorption film 14 was set to 70% or more. With this arrangement, transmitted light of 25% or higher is obtained even with use of a four-layer structure, and the deodorizing device was sufficiently functioned, even taking into account the light loss. In the multi-layer structure, it is desirable to obtain a transmittance of at least 50% or higher for one purification plate.

The embodiment and the modifications of the invention have been described as above. The invention is not limited to the foregoing embodiment and modifications, and the embodiment of the invention may be modified in various ways other than the above.

For instance, in the embodiment of the deodorizing device, the light to be emitted from the LEDs 61 is pulse light or DC light. Alternatively, the LEDs 61 may be controlled in such a manner that the light emission energy of the LEDs 61 is stepwise changed depending on a detection signal from the odor sensor 51. The modification is more advantageous in efficiently causing a photocatalytic reaction, because the light energy reaching the photocatalyst film 13 can be changed depending on the amount of a material to be purified that is contained in the purification region 20c.

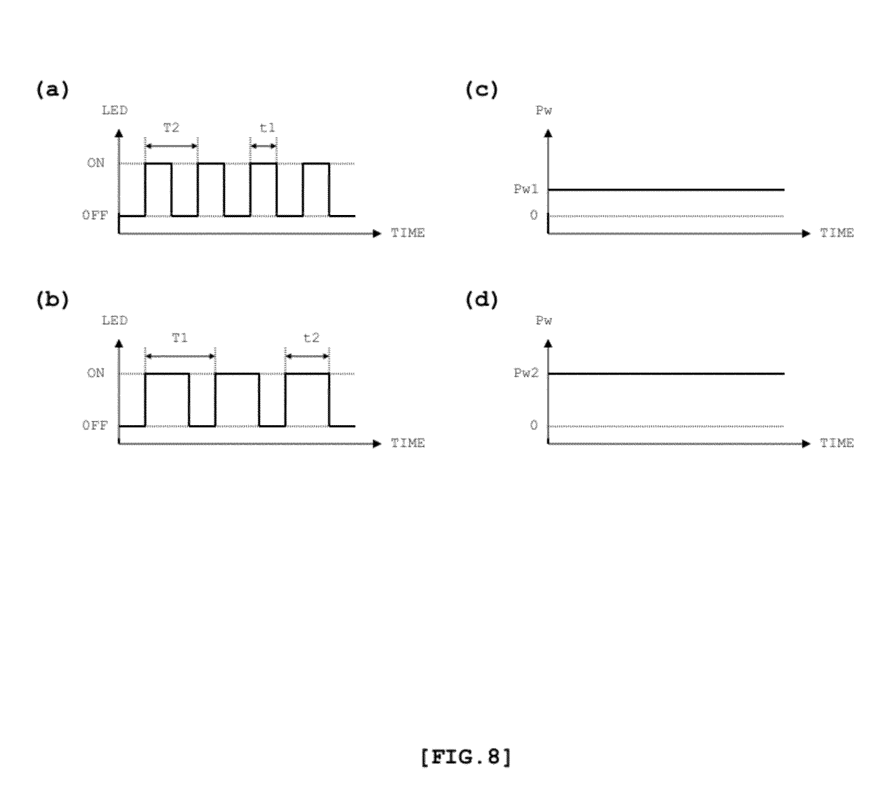

In order to change the light emission energy of the LEDs 61, the duty ratio of the LEDs 61 may be changed. For instance, as shown in FIG. 8(a), the LEDs 61 may be caused to emit light at a cycle T2, which is different from the cycle T1 shown in FIG. 5(c). Further alternatively, as shown in FIG. 8(b), the LEDs 61 may be caused to emit light with an emission time, which is different from the emission time t1 shown in FIG. 5(c).

Further, in the embodiment, the LEDs 61 are used as a light source for causing a photocatalytic reaction. Alternatively, a semiconductor laser may be used in place of the LEDs 61. In the modification, the light energy to be irradiated onto the photocatalyst film 13 is adjusted by switching the semiconductor laser between DC emission and pulse emission, as described in the embodiment. Alternatively, as shown in FIG. 8(c), 8(d), the light energy to be irradiated onto the photocatalyst film 13 may be adjusted by switching the emission power of the semiconductor laser between Pw1 and Pw2. Further alternatively, in the case where the light emission energy of the semiconductor laser is stepwise changed depending on a detection signal from the odor sensor 51, the emission power Pw of the semiconductor laser may be stepwise changed. In particular, the semiconductor laser is advantageous to change the emission energy stepwise, because the semiconductor laser has a wide emission power range.

The embodiment of the invention may be changed or modified in various ways as necessary, as far as such changes and modifications do not depart from the scope of the claims of the invention hereinafter defined.

### Reference Signs List

10, 110, 120 . . . purification plate (photocatalytic structural member)

11 . . . substrate

12 . . . transmissive film

13 . . . photocatalyst film

14 . . . adsorption film

31, 32 . . . fan

51, 52 . . . odor sensor

60, 130 . . . light emission unit (light source)

61 . . . LED (light source)

1. A photocatalytic structural member, comprising:
a substrate having a concave-convex structure formed on a surface thereof; and
a photocatalyst film which is disposed on a side of the concave-convex structure of the substrate and reflects a shape of the concave-convex structure, wherein
the concave-convex structure is formed with a pitch smaller than a wavelength of light which causes a photocatalytic reaction on the photocatalyst film.

2. The photocatalytic structural member according to claim 1, further comprising:
a transmissive film disposed between the substrate and the photocatalyst film.

3. The photocatalytic structural member according to claim 1, further comprising:
an adsorption film which is disposed on the photocatalyst film and traps a material for contacting the material with the photocatalyst film.

4. A deodorizing device, comprising:
a photocatalytic structural member including a substrate having a concave-convex structure formed on a surface thereof, and a photocatalyst film which is disposed on a side of the concave-convex structure of the substrate and reflects a shape of the concave-convex structure, the concave-convex structure being formed with a pitch smaller than a wavelength of light which causes a photocatalytic reaction on the photocatalyst film;
a light source which emits the light from a side of the substrate to the photocatalytic structural member; and
a fan which causes an air to flow to the photocatalytic structural member.

5. The deodorizing device according to claim 4, wherein
the light source emits the light at a first power when the deodorizing device is activated, and
the light source emits the light at a second power larger than the first power, after a lapse of a predetermined time from the time when the deodorizing device is activated.

6. The deodorizing device according to claim 4, further comprising:
an odor sensor which is disposed in an air passage for causing the air to flow to the photocatalytic structural member, wherein
an emission power of the light from the light source is controlled based on a detection signal from the odor sensor.

7. The deodorizing device according to claim 4, further comprising:
a transmissive film disposed between the substrate and the photocatalyst film.

8. The deodorizing device according to claim 4, further comprising:
an adsorption film which is disposed on the photocatalyst film and traps a material for contacting the material with the photocatalyst film.

9. The deodorizing device according to claim 4, further comprising:
an odor sensor which is disposed in an air passage for causing the air to flow to the photocatalytic structural member, wherein
the light source emits the light at a first power in the case where a detection signal from the odor sensor is equal to or smaller than a predetermined value, and the light source emits the light at a second power larger than the first power in the case where the detection signal from the odor sensor is larger than the predetermined value.

10. The deodorizing device according to claim 4, further comprising:
a first odor sensor and a second odor sensor which are respectively disposed on an upstream side and a downstream side of the photocatalytic structural member, wherein
the fan is driven at a first rotation number and the light source emits the light at a first power, when the deodorizing device is activated; and the fan is driven at a second rotation number larger than the first rotation number and the light source emits the light at a second power larger than the first power, in the case where a detection signal from the first odor sensor is larger than a detection signal from the second odor sensor after the activation of the deodorizing device.
